SEQUENCE LISTING (18 sequences)

SEQ ID NO: 1 - DNA, 24 bp, Artificial sequence
  Primer
tttttagaggatttgagggatagg

SEQ ID NO: 2 - DNA, 24 bp, Artificial sequence
  Primer
ctacctaattccaattcccctaca

SEQ ID NO: 3 - DNA, 14 bp, Homo Sapiens
acmgtccatcmgct

SEQ ID NO: 4 - DNA, 14 bp, Homo Sapiens
acmgtuuatcmgut

SEQ ID NO: 5 - DNA, 12 bp, Artificial sequence
  Primer
acgtccatcgct

SEQ ID NO: 6 - DNA, 12 bp, Artificial sequence
  Primer
augtuuatugut

SEQ ID NO: 7 - DNA, 60 bp, Homo Sapiens
gaggcmgccmgccmgccmgccmgctgccmgccmgcacactgggatccmgctcmggcagca

SEQ ID NO: 8 - DNA, 42 bp, Homo Sapiens
cgaacgaatcccaatatacgacgacaacgacgacgacgacgc

SEQ ID NO: 9 - DNA, 42 bp, Homo Sapiens
cgccgccgccgccgctaccgccgcacactaaaatccgctcga

SEQ ID NO: 10 - DNA, 60 bp, Homo Sapiens; identical to SEQ ID NO: 7

SEQ ID NO: 11 - DNA, 42 bp, Homo Sapiens
caaacaaatcccaatatacaacaacaacaacaacaacaacac

SEQ ID NO: 12 - DNA, 42 bp, Homo Sapiens
caccaccaccaccactaccaccacacactaaaatccactcaa

SEQ ID NO: 13 - DNA, 126 bp, Homo Sapiens
tggttaaggttattggggtgtttttggagatttaggggttaattggttggtgtttatatttatttgtggggattagtgttgtggtggagaagagtaatagtagaagttggagttggagtttgggag

SEQ ID NO: 14 - DNA, 115 bp, Homo Sapiens
cractccracttctactattactcttctccrccrcracactaatccccacaaataaatataaacaccraccrattaacccctaaatctccraaaacaccccaataaccttaacca

SEQ ID NO: 15 - DNA, 32 bp, Artificial sequence
  Primer
gttgtgtaattcgttggatgcggattagggcg

SEQ ID NO: 16 - DNA, 32 bp, Artificial sequence
  Primer
cgccctaatccgcatccaacgaattacacaac

SEQ ID NO: 17 - DNA, 15 bp, Artificial Sequence
  Primer
acgacggccaaattt

SEQ ID NO: 18 - DNA, 12 bp, Artificial Sequence
  Primer
tgctgccggttt

(19) United States
(12) Patent Application Publication
(10) Pub. No.: US 2018/0187246 A1
(43) Pub. Date: Jul. 5, 2018

(54) PROBES AND A METHYLATION IN SITU HYBRIDIZATION ASSAY

(71) Applicant: UNIVERSITEIT GENT, Gent (BE)

(72) Inventors: Marusya Lieveld, Gavere (BE); Wim Van Criekinge, Waarloos (BE); Davy Vanden Broeck, Leest (BE)

(73) Assignee: Universiteit Gent, Gent (BE)

(21) Appl. No.: 15/740,255

(22) PCT Filed: Jul. 12, 2016
(86) PCT No.: PCT/EP2016/066510

§ 371 Date: Dec. 27, 2017

(30) Foreign Application Priority Data
Jul. 15, 2015 (EP) 15176744.9

(51) Int. Cl. C12Q 1/6827

(52) U.S. Cl. CPC C12Q 1/6827; C12Q 2525/161; C12Q 1/686; C12Q 2565/102; C12Q 2523/125; C12Q 2537/163

(57) ABSTRACT

The disclosure relates to the field of molecular pathology (for example, cancer diagnosis, prognosis, treatment and/or therapy prediction) through the detection of RNA, mutations, copy number changes and determination of the methylation status of specific sequences of the genome of individual patients in hybridization assays (southern blot, ISH, dot blot) including in situ determination of the methylation status of specific sequences of the genome of individual patients in individual cells. More specifically, this disclosure relates to: a) target-specific probes covalently attached to a labeled tail, b) the synthesis method of said the probe, c) the usage of said the probe such as an in situ hybridization-based method to correlate the methylation status of a promoter region of a gene in a biopsy or cytology specimen of a patient to the morphology and localization in that specimen, and d) kits comprising the target-specific probes. The latter method and products allow detection of (epi) genetic changes in specific cell types of histological or cytological (cancer) specimens or on membranes that will contribute to scientific research and that will help physicians to accurately diagnose diseases and/or start an appropriate treatment.

### CROSS-REFERENCE TO RELATED APPLICATIONS

This application is a national phase entry under 35 U.S.C. § 371 of International Patent Application PCT/EP2016/066510, filed Jul. 12, 2016, designating the United States of America and published in English as International Patent Publication WO 2017/009322 A1 on Jan. 19, 2017, which claims the benefit under Article 8 of the Patent Cooperation Treaty to European Patent Application Serial No. EP15176744.9, filed Jul. 15, 2015.

### TECHNICAL FIELD

This application relates to the field of molecular pathology (for example, cancer diagnosis, prognosis, treatment and/or therapy prediction) through the detection of RNA, mutations, copy number changes and determination of the methylation status of specific sequences of the genome of individual patients in hybridization assays (southern blot, ISH, dot blot) including in situ determination of the methylation status of specific sequences of the genome of individual patients in individual cells. More specifically, this disclosure relates to: a) target-specific probes covalently-attached to a labeled tail, b) the synthesis method of the probe, c) the usage of the probe such as an in situ hybridization-based method to correlate the methylation status of a promoter region of a gene in a biopsy or cytology specimen of a patient to the morphology and localization in that specimen, and d) kits comprising the target-specific probes. The latter method and products allow detection of (epi) genetic changes in specific cell types of histological or cytological (cancer) specimens or on membranes that will contribute to scientific research and that will help physicians to accurately diagnose diseases and/or start an appropriate treatment.

### BACKGROUND

The following target-specific probes or signal amplification systems for in situ hybridization have been described:

### 1) Padlock Probe—Rolling Circle Amplification (RCA) (Larsson et al., 2004):

This method combines PCR amplification for sufficient signal amplification and enzymatic restriction to allow probe access; and does thus not allow quantification of the target at physiological levels.

In RCA, the target DNA is restriction digested at the 3′ end of the target sequence and irreversibly made single-stranded by strand-specific 5′-3′ exonucleolysis. Padlock probes are hybridized to their target sequences and the probe ends are joined through ligation, locking the probe onto the target molecule. After ligation, the RCA is initiated by the F29 DNA polymerase by turning the target molecule into a primer through 3′-5′ exonucleolysis of any 3′ end protruding beyond the padlock probe hybridization site. The padlock probe then serves as the template for DNA synthesis. The RCA product is detected through hybridization of fluorescence-labeled oligonucleotides to tag sequences, specific for the padlock probe (Larsson et al., 2004).

The sensitivity of this technique is only 10% since the enzymatic restriction step that exposes the sense or anti-sense strand is not absolute and is difficult to regulate; further factors contributing to the reduced sensitivity is the low PCR efficiency and DNA loss.

RCA is thus based on amplification of the target-specific probe and detection of the amplified material.

Moreover, the detection probes used in RCA are molecular inversion probes (MIP).

The probes are designed with complementary sequences to the target at its 5′ and 3′ ends. The internal region contains two universal PCR primer sites that are common to all MIPs, as well as a probe-release site, which is usually a restriction site.

### 2) Lollipop Probes for Signal Amplification (U.S. 2002/0192658)

A lollipop oligomer is a branched oligomer that comprises a tail portion, a right arm portion, and a left arm portion. The two arms each end with sequences complementary to adjacent sequences in a target sequence. This allows the right and left arms to be ligated together when the oligomer is hybridized to the target sequence. The tail portion comprises a rolling circle replication primer. Amplification of the signal is then performed by means of RCA. The tail portion can then be detected at the location of the target sequence.

3) Branched DNA Amplification (bDNA) (Collins et al., 1997):

The bDNA protocol includes four probe hybridization steps followed by sufficient washing after each step. First, a target-specific probe containing a small toe is added to the sample followed by a second hybridization with a pre-amplifier oligo that will bind the target-specific probe. A third hybridization with the amplifier probe that will bind the pre-amplifier is then performed. Finally, labeled probes that will hybridize with the amplifier are added. The bDNA system is based on a four-step hybridization protocol to create an amplification tree that gives sufficient signal for target probe detection. On the contrary, the probe of this disclosure is a one-molecule probe that allows target detection in a “one hybridization step” protocol.

bDNA is composed of four single-stranded oligomers that hybridize with each other to create an amplification tree. The probe of this disclosure consists of one molecule that contains a target-specific part and a signal amplification part.

A specific binding of the branched molecules in bDNA is prevented by including isobases in their sequences.

### 4) Tyramide Signal Amplification (TSA) System (Schriml et al., 1999):

TSA is an enzyme-mediated detection method that uses horseradish peroxidase (HRP) for signal amplification. In this system, biotin-labeled probes are hybridized with the target following addition of streptavidin-HRP. Tyramide-fluorophore are deposited by HRP in the amplification reaction.

Signal amplification of target-specific probes is performed in two steps and is generated by an enzymatic process (deposition of labeled tyramide), whereas this disclosure relates to signal amplification by means of a strongly labeled signal tail sequence linked to a target-specific part.

Probes used for TSA amplification, contrary to the probes of this disclosure, do not contain a signal amplification part.

Overall drawbacks of the above-mentioned alternative methods and probes are their complex and expensive protocols, low sensitivity, low quantification possibilities, various signal amplification steps after probe hybridization, and extensive washing steps, resulting in target lost and a high background staining, which hamper their use for routine application. Thus, there is a need to design better performing probes that are capable of visualizing targets in hybridization assays and that can be used for routine application.

5) The Following Methods that may Allow Detection of Methylation changes have been Described:

5.1 Nuovo et al. (Nuovo et al., 1999) disclose methylation-specific PCR in situ hybridization (ISH). They monitored p16^{INK4a} methylation changes in Formalin-Fixed, Paraffin-Embedded (FFPE) tissue samples. In this protocol, in situ bisulfite conversion is first performed overnight, followed by methylation-specific in situ PCR (MSP). MSP uses primers specific for detection of sequence differences between methylated versus unmethylated DNA, that result from bisulfite modification; Bisulfite modified DNA was amplified with p16^{INK4a} gene-specific primers 5′-TTTTTAGAGGATTTGAGGGATAGG-3′ (sense, SEQ ID NO:1) and 5′-CTACCTAATTCCAATTCCCCTACA-3′ (anti-sense, SEQ ID NO:2). After amplification, in situ hybridization was performed by simultaneously adding long (>80 base pairs (bp) sized) unmethylated-specific or methylated-specific internally digoxigenin-labeled probes. Here again, PCR amplification is used for signal amplification and the efficiency of the test relies on PCR specificity followed by amplicon detection by probes. Thus, this test does not allow detection of the target at physiological levels. Moreover, because the target is amplified and the amplicons will crowd the nuclei, co-localization of multiple targets (for example, an unmethylated target and a methylated target) will be very difficult to interpret.

5.2 Larsson et al. (Larsson et al., 2004) describe padlock probes for single nucleotide polymorphisms (SNPs) detection. These are oligonucleotide probes that induce circularization of the target after hybridization to the target region. Double-stranded (dsDNA) is made accessible for padlock probe hybridization by enzymatic digestion. A combination of restriction enzymes and exonuclease enzymes (MSCI and EcoRV) is used. Following PCR amplification, labeled oligo probes are added and these recognize the amplified target. However, the efficiency of the probe hybridization is only 10% because enzymatic restriction that should expose the sense or anti-sense strand is not absolute and it is difficult to regulate. Further factors are PCR efficiency and DNA loss. This method thus combines PCR amplification for sufficient signal amplification and enzymatic restriction to allow probe access and, thus, does not allow quantification of the target at physiological levels.

5.3 Li et al. (Li et al., 2013) disclose microscopic evaluation of the methylation status at satellite repeats. This paper demonstrates the detection of the methylation status of minor and major satellite repeats using labeled Locked Nucleic Acids (LNA) probes. Probe recognition depends on cross-linking of a bipyridine-adenine derivative at the position corresponding to the methylated cytosine in the presence of osmium; therefore, the described method does not allow detection of unmethylated sequences, and so hypomethylation cannot be observed. The described method can only be used for detection of highly abundant repeats, because these small probes cannot compete with re-hybridization of the complementary strands and will not generate enough signals for microscopic evaluation of single copy genes.

In order to ensure a sufficient sensitivity for microscopic evaluation, the above-described methods must either be PCR-based (Larsson et al., 2004; Nuovo et al., 1999) or they can only detect abundant targets such as satellite repeats (Li et al., 2013). Specificity is achieved by target-specific amplification with methylation-specific primers, following in situ hybridization with >80 bp probes (Nuovo et al., 1999), cross-linking by means of a bipyridine-adenine derivative at the position corresponding to the methylated cytosine in the presence of osmium (Li et al., 2013) or ligation of padlock probes at SNP positions (Larsson et al., 2004).

### BRIEF SUMMARY

The disclosure relates to a (single-stranded) probe comprising at least the following parts: a) a first part comprising a nucleotide sequence that is (semi-)complementary to a target sequence and is similar to a reverse primer, b) a second part functioning as a spacer and comprising at least one nucleotide that is not complementary to the target sequence, and c) a third part comprising a nucleotide sequence that is not complementary to the target sequence, wherein the nucleotides are composed of only three different types of nucleotides chosen from the five different types A, C, G, T or U and wherein 10% to 100% of the nucleotides are labeled.

The disclosure further relates to a probe as defined above further comprising a fourth part comprising a nucleotide sequence that is not complementary to the target sequence but is (semi-)complementary to a forward primer.

The disclosure further relates to a probe as defined above further comprising a fifth part comprising the types of nucleotides that are not chosen in the third part.

The disclosure also relates to a probe as defined above wherein the third part is made double-stranded (double-stranded probe) by PCR or by hybridization with a (semi-)complementary sequence to the signal tail.

The disclosure also relates to a process to synthesize a single-stranded) probe as defined above comprising:
a PCR step that is performed in the presence of a reverse primer that is similar to the first part of the probe, a mix of labeled and unlabeled dNTPs in order to synthesize the third part of the probe as defined above and a template comprised of the following parts: 1) optionally, a first part comprising a nucleotide sequence that is similar to a forward primer, 2) a second part comprising a nucleotide sequence composed of only three different types of nucleotides chosen from the five different types A, C, G, T or U, 3) optionally, a third part comprising the types of nucleotides that are not chosen in the second part, 4) a fourth part functioning as a spacer and comprising at least one nucleotide, and 5) a fifth part comprising a nucleotide sequence that is (semi-)complementary to a reverse primer.

The disclosure further relates to a process of synthesizing a (double-stranded) probe as defined above comprising:
a PCR step as defined above wherein the template comprises the first part and/or fifth part, and a second PCR step, which is performed in the presence of a forward primer, a dNTP mix is included that only contains nucleotides that are complementary to the three different types of nucleotides chosen in the second part of the template according to the first PCR step.

The disclosure relates to a process of synthesizing a double-stranded probe by hybridization of the signal tail to a (semi-)complementary sequence to the signal tail.

The disclosure also relates to the usage of a probe as defined above to specifically detect small target sequences.

The disclosure further relates to a kit comprising a probe as defined above.

The disclosure further relates to a method of detecting methylation change-induced single nucleotide polymorphism in situ and/or to distinguish methylation heterogeneity from hemi-methylation and mono-allelic methylation in a sample taken from a patient comprising:
obtaining a sample from the patient,
treating the sample with adequately dosed pepsin and/or protease K and/or HCL and/or detergent and/or ethanol to permeabilize samples and to remove proteins from the sample,
incubating the sample with adequately dosed bisulfite reagents in the presence of a RNase inhibitor to create non-complementary single-stranded DNA strands,
incubating the samples with specifically designed blocking probes and/or DNA-protecting probes for at least 15 minutes, and
incubating the sample with specifically designed target-specific probes.

The disclosure relates to a method as defined above wherein the target-specific probes are probes as defined above.

The disclosure further relates to a kit comprising a target-specific probe and/or blocking probes and/or DNA protecting probes as defined above.

The disclosure finally relates to the usage of a kit as defined above to perform the method as defined above.

### BRIEF DESCRIPTION OF THE DRAWINGS

FIG. 1: Schematic representation of target-specific probe consisting of a target-specific compound (thick bar) and a labeled compound (thin bar, light grey and stars). The labeled compound can be sealed by an unlabeled semi-complementary or complementary sequence (thin bar dark grey).

FIG. 2a: A sealed, target-specific probe or Uniprobe Signal Amplification System (UPSAS) of the disclosure consists of six major parts: a) a target-specific probe sequence with a similar sequence as the reverse primer (RP), b) a spacer of at least one nucleotide, c) an A-stretch of at least one nucleotide (this is included to stop probe sealing when probe sealing is performed with three nucleotides), d) a labeled part that consists of UGC or TGC nucleotides (this is the signal tail), e) a sequence that is (semi-)complementary to the forward primer used in probe synthesis, and f) a “seal” consisting of a (semi-)complementary sequence of the RP and a stretch of AGC nucleotides. FIG. 2b) An unsealed UPSAS probe consisting of four major parts: a) a target-specific probe sequence with a similar sequence as the reverse primer, b) one or more spacers of at least one nucleotide (may also include an A-stretch as indicated in FIG. 2a), c) a signal tail that consists of UGC or TGC nucleotides, and d) a sequence that is (semi-)complementary to the forward primer used in probe synthesis (optional).

FIGS. 3a and 3b: probe synthesis of UPSAS.

FIG. 3a) PCR 1: During the first step, PCR 1 is performed with a forward primer, a reverse primer, a probe template and labeled nucleotides (ATTO, FITC, fast red, biotin or others). The probe template consists of five major parts (from 5′ end to 3′ end): 1) a sequence that is similar to the forward primer used in probe synthesis and sealing, 2) a part that consists of AGC nucleotides (template for signal tail), 3) a T-stretch consisting of at least one nucleotide (this is included in the template to stop probe sealing in PCR2). Probe sealing is performed in the absence of dUTP/dTTP, blocking elongation of the seal at the site of the repeat), 4) a spacer of at least one nucleotide, and 5) a sequence that is (semi-)complementary to the reverse primer and thus to the target-specific probe (see sample SEQ ID NOS:17-18). After the first PCR run, a single-stranded labeled probe is generated of which the reverse primer now constitutes the detection probe part of UPSAS.

FIG. 3b) PCR 2: Optional step to create a “sealed” probe: The second PCR step (PCR 2), is called the “probe sealing” step: During the probe sealing step, primer elongation of the single-stranded labeled probe is performed with only one primer (forward primer) in the presence of labeled nucleotides. After PCR2, a partially double-stranded probe is generated with the detection probe still free for target recognition and binding. Elongation of forward primer is blocked at the A-repeat at the 3′ end of the target-specific probe part because probe sealing is performed with a dNTP-mix consisting of dATP, dGTP, dCTP and lacking dUTP/dTTP.

FIGS. 4a-4f: Detection of GSTP1 hypermethylation with UPSAS in cell lines. Fluorescence microscopic evaluation of GSTP1 hypermethylation in MCF7 (FIG. 4a), LNCaP (with sealed and unsealed UPSAS probes) (FIG. 4b), BT474 (FIG. 4c), SKBR3 (FIG. 4d), MDA-MD-231 cell lines (FIG. 4e) and a PC3 cell line (FIG. 4f) not treated with bisulfite. MCF7, LNCaP (with sealed and unsealed UPSAS probe), SKBR3 and BT474 cells show two spots per cell, indicating GSTP1 hypermethylation of both alleles. The MDA-MD-231 cell line and PC3 cell line not treated with bisulfite show no signals, indicating the absence of the GSPT1 hypermethylated target.

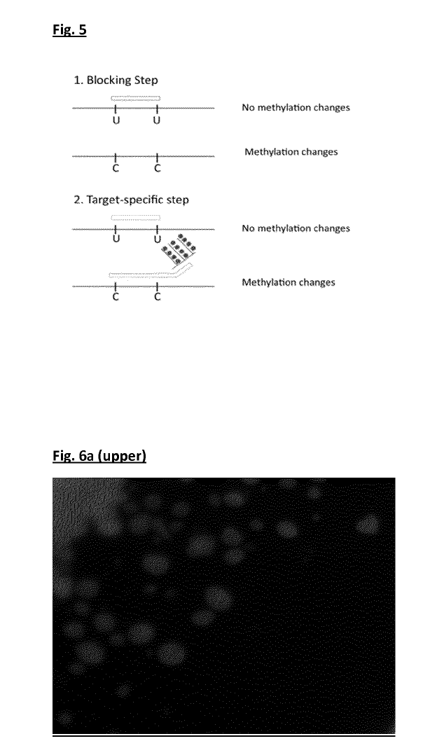

FIG. 5: The methylation in situ hybridization (MISH) assay. First homologous regions to the target region are blocked by blocking probes. In step 2, target-specific probes are added. Target-specific probes are detected by means of a labeled compound that can be covalently linked to the probe or is linked through hybridization. Blocking and hybridization with the target-specific probes may also be performed in one single step.

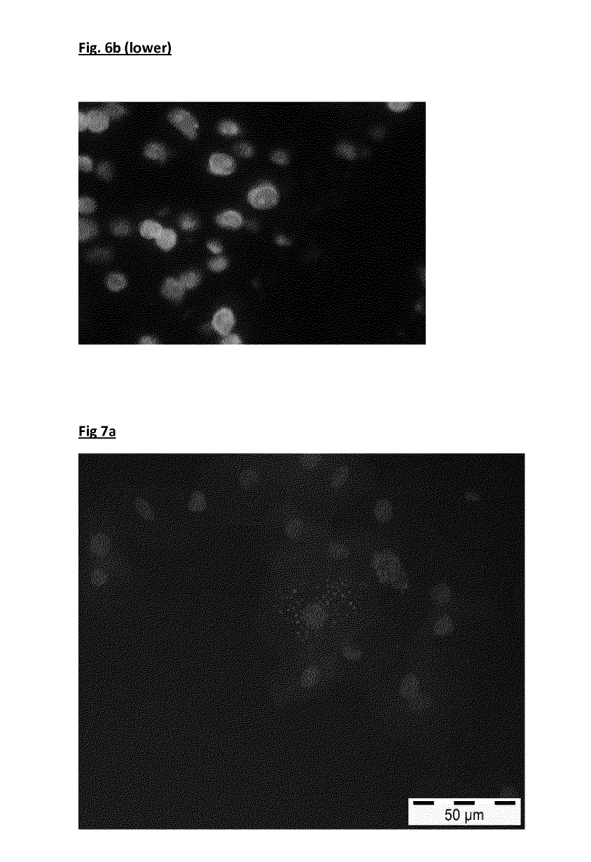

FIGS. 6a and 6b: Fluorescence microscopic evaluation of cell adhesion molecule 1 (CADM1 (Overmeer et al., 2008)) hypermethylation in CADM1-positive SiHa cell line (cervical cancer cell line) (FIG. 6b—lower picture) and CADM1-negative skin cells (FIG. 6a—upper picture). SiHa cells show two spots per cells while skin cells do not show any signal.

FIGS. 7a-7d: Fluorescence microscopic detection of HPV73 mRNA in monolayers of two HPV73-positive cervical specimens using one L1 (small green dots) and two E1 (larger green dots) specific UPSAS probes (FIGS. 7a, 7b (specimen 1), 7c and 7d (specimen 2)).

### DETAILED DESCRIPTION

The disclosure includes, in the first instance, of a target-specific amplification probe called the Uniprobe Signal Amplification System (UPSAS), which targets and visualizes mutations and methylation changes in in a patient's DNA and/or detects RNA in hybridization assays (dot blot, southern blot, ISH) and its synthesis.

This disclosure thus relates to a target-specific amplification probe to detect target RNA and/or DNA sequences in hybridization assays, wherein the probe is characterized by:
its nature as a nucleic acid or nucleic acid analog consisting of a small target-specific detection probe that is able to specifically detect RNA or DNA target sequences, and is covalently attached to a “signal tail,” which is a nucleic acid or nucleic acid analog of which the sequence only consists of three different types of nucleotides (TGC, for example) instead of the usual four different types of nucleotides (TGCA) and shows no sequence complementarity to the human genome or to the bisulfite-converted sequence of the human genome and contains 10% to 100% fluorescent labeled nucleotides.

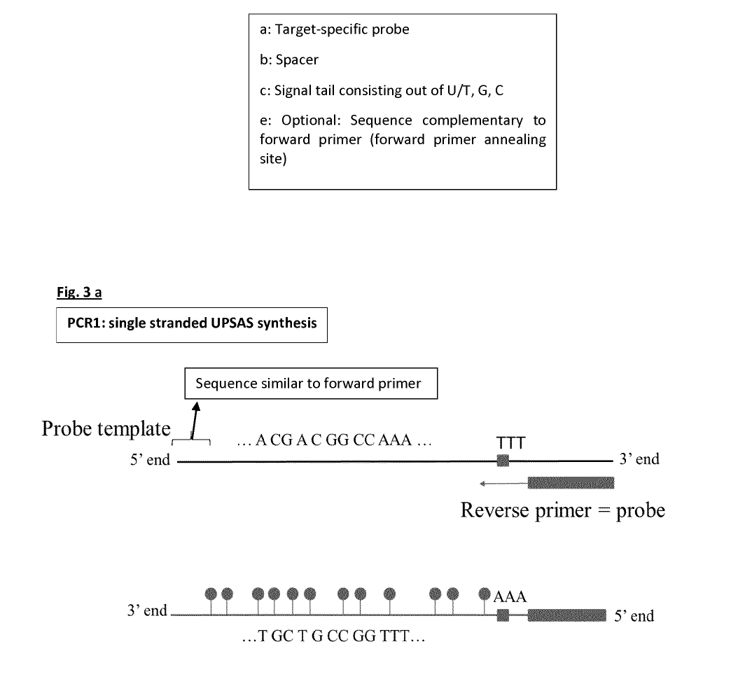

In other words, this disclosure relates to a probe consisting of four major parts: a) a target-specific probe sequence with a similar sequence as the reverse primer, b) optionally, one or more spacers of at least one nucleotide (which may also include a stretch of single-type nucleotides such as an A-stretch as indicated in FIGS. 2a and 2b), c) a signal tail that consists of three different types of nucleotides excluding the type of nucleotide of the latter stretch, which is part of the spacer (such as UGC or TGC nucleotides), and d) optionally, a sequence that is (semi-)complementary to a forward primer.

The disclosure also relates to a method that allows direct targeting and visualizing of methylation changes in single copy genes in patients' DNA and RNA in situ in one step using specifically designed target-specific probes linked with a labeled compound that contains a vast number of chromogens or fluorescent dyes, to blocking probes and to DNA protecting probes.

In other words, this disclosure provides a non-PCR-based method that directly detects methylation changes in patients' DNA or RNA by incorporating a large signal-generating compound (labeled compound or signal tail) at the target-specific part of the target-specific probes rather than replicating target sequences for sufficient detection. Therefore, this disclosure allows for detection of small targets including methylation changes at physiological levels.

This disclosure further relates to the “target-specific probes,” wherein:
their nature as a nucleic acid or nucleic acid analog, consisting of a small target-specific part, which is able to distinguish nucleotide polymorphisms and methylation changes, and is bound to one or more labeled compound(s), which is a nucleic acid or nucleic acid analog of which the sequence(s) shows no sequence complementarity to the human genome or to the bisulfite converted sequence of the human genome and contains 10% to 100% fluorescent-labeled nucleotides.

More specifically, UPSAS is characterized by:
a. Its nature as a nucleic acid or nucleic acid analog,
b. Its comprehensive structure comprising:
1. A target-specific detection probe sequence at the 3′ or 5′ end, preferentially at the 5′ end.
2. At the 3′ or 5′ end, preferentially at the 3′ end of the sequence of the detection probe part, one or more spacers are included that ensures that the signal amplification part does not sterically interfere with probe binding. One of the spacers may also include a stretch of single-type nucleotides, comprising the types of nucleotides that is excluded in the signal tail.
3. At the 3′ or 5′ end, preferentially at the 3′ end of the spacers, a signal tail between 10 bp and 100 kbp and, preferentially between 100 bp and 10 kbp, is included. The signal tail contains 10% to 100% fluorescent-labeled nucleotides.
4. At the 3′ or 5′ end, preferentially at the 3′ end of the signal tail, a forward primer recognition site is included. The primer recognition site is used as a primer annealing site during probe sealing (the signal tail is made double-stranded in a second PCR reaction to create sealed UPSAS; probe sealing is performed to reduce specific binding and/or for incorporation of additional labels) but it is also used in the first PCR reaction for probe synthesis (to aim an exponential increase of the amount of probe).
5. (Semi-)complementary (only for sealed UPSAS) to the signal tail, a labeled or unlabeled nucleotide strand (“seal”) may be included. By making the signal tail double-stranded, aspecific binding of the signal tail and thus mistargeting of the probe is avoided.

The target-specific detection probe part of UPSAS is further specifically characterized by:
1. Its code: it should be (semi-)complementary (complementary or semi-complementary) to the sequence of the target sequences, through Watson-Crick base pairing. For example, if the sequence of the methylation marker for a specific disease is 5′ -GTT GTG TAA TTC GTT GGA TGC GGA TTA GGG CG-3′ (SEQ ID NO:15), ideally, the target-specific probes for the sense and anti-sense strand should, respectively, be 5′-CGC CCT AAT CCG CAT CCA ACG AAT TAC ACA AC-3′ (SEQ ID NO:16).
2. Their size: the target-specific part of the probe is between 8 bp and 10 kbp and, preferentially, between 14-200 bp.

The “signal tail” is specifically characterized by:
1. Its nature as a nucleic acid or nucleic acid analog
2. Its structure:
   a. Its size: the signal tail is between 10 bp and 100 kbp and, preferentially, between 100 bp and 10 kbp.
   b. The signal tail consists of 10%-100% bases modified with chromogens or fluorescent dyes (for instance, ATTO, biotin, FITC, Alexa Red and/or others).
   c. For sealed UPSAS: the signal tail is (partially) made double-stranded with a “seal” to prevent aspecific binding of UPSAS. Hereupon, the seal may also contain labeled nucleotides and may thus be used to incorporate additional labels in the probe.
3. Its code: remaining aspecific binding of the signal tail is prevented by the nature of its sequence composition: the signal tail mainly consists of three nucleotides (for example, U/TGC if one spacer consists of a “stretch” of A nucleotides or, U/TGA if one spacer consists of a “stretch” of C nucleotides or, U/TAC if one spacer consists of a “stretch” of G nucleotides or, AGC if one spacer consists of a “stretch” of T nucleotides) instead of the “usual” four nucleotides (TGCA).

The “spacers” between the signal tail and the detection probes are specifically characterized by:
1. Its nature as a nucleic acid or nucleic acid analog
   2. Its structure (FIG. 2a):
   a. For sealed UPSAS:
      i. The first spacer next to the signal tail is, for example, an adenine-repeat. It is composed of a stretch of 1-10,000 and, preferentially, of 1-200 A, C, G, T or U.
      ii. Optionally, additional spacers of 1-10,000 and, preferentially, of 1-200 nucleotides are included next to the A-stretch. The sequences of these spacers show no complementarity to the sequences flanking the target region in order to prevent potential interference of the spacers with probe binding.
   b. For unsealed UPSAS:
      i. Spacers of 1-10,000 and, preferentially, of 1-200 nucleotides are included next to the target-specific probe part. The sequences of these spacers show no complementarity to the sequences flanking the target region in order to prevent potential interference of the spacers with probe binding.

An annealing site for the forward primer is included at one end (preferentially at the 3′ end) of the UPSAS probe. The forward primer annealing site is characterized by:
a. Its nature as a nucleic acid or nucleic acid analog.
b. Its size: between 8 bp and 10 kbp and, preferentially, between 14-200 bp.
c. Its (semi-)complementary to the forward primer used for probe synthesis and probe sealing.

The forward primer annealing site is mandatory for the synthesis of sealed UPSAS in the case where the signal tail is made double-stranded by PCR to reduce aspecific binding and/or for incorporation of additional labels, but optional for the synthesis of unsealed UPSAS or in the case where the sealed probe is made double-stranded by hybridization with the seal.

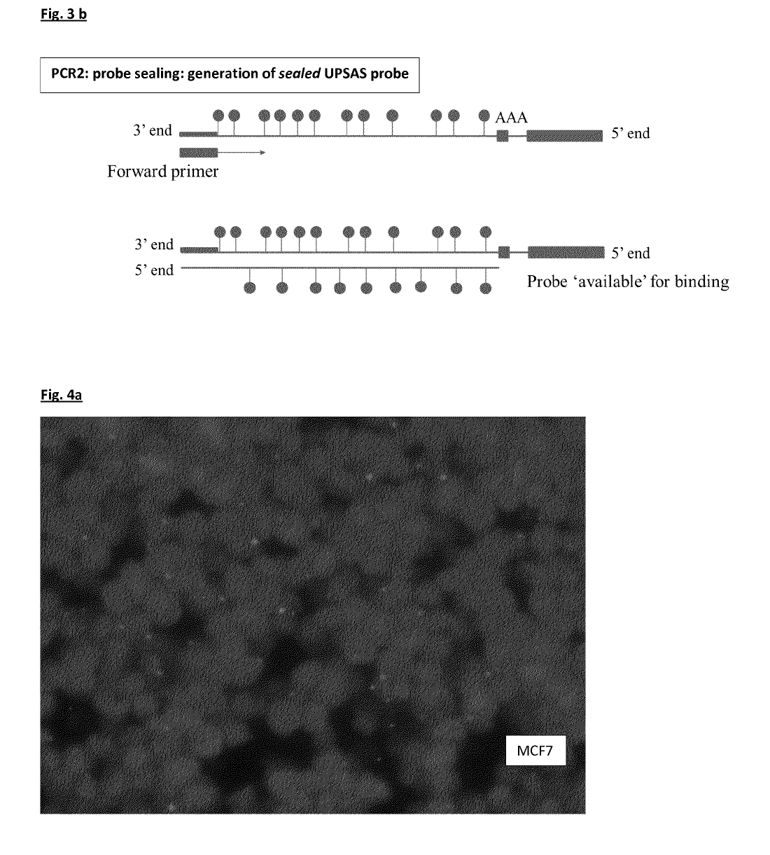

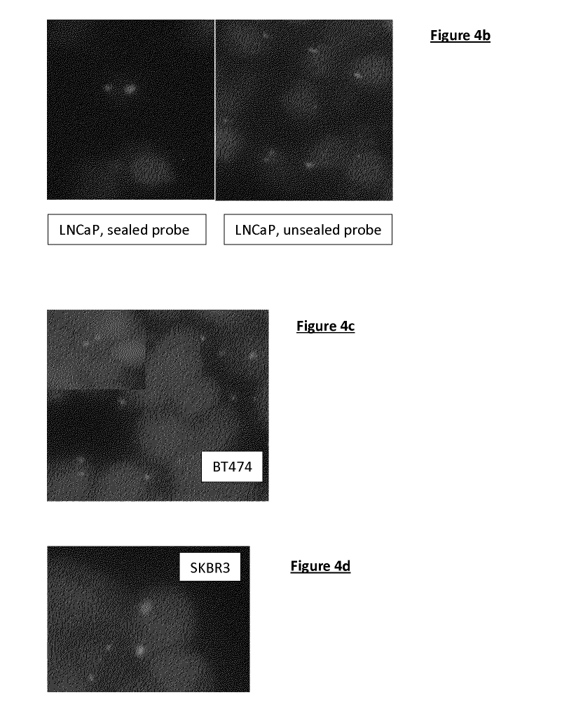

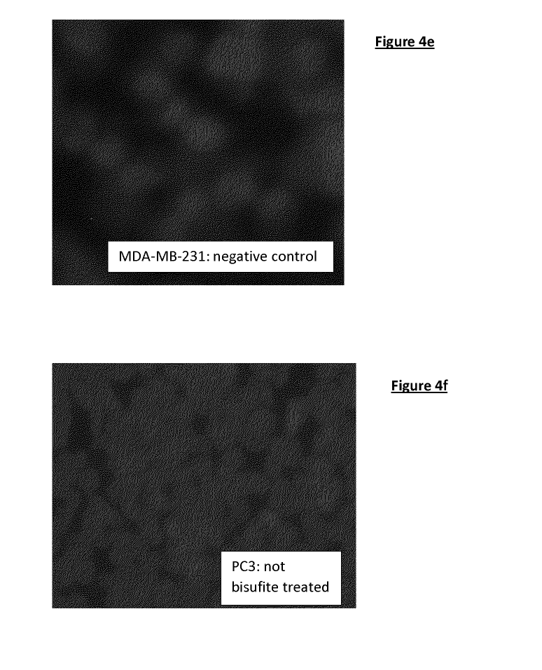

Synthesis of UPSAS is performed in one (unsealed UPSAS) or two PCR steps (sealed UPSAS) (FIGS. 3a and 3b): FIG. 3a: During the first step, PCR is performed with a forward primer (optional for unsealed UPSAS or when sealed UPSAS is created by hybridization with the seal, mandatory for sealed UPSAS created by PCR), a reverse primer (the reverse primer will constitute the target detection probe), a probe template and labeled nucleotides (ATTO, FITC, fast red, biotin and/or other labels). After the first PCR, a single-stranded labeled probe is generated.

FIG. 3b: The second PCR step is called the “probe sealing” step (only required for PCR-sealed UPSAS synthesis):

During the probe sealing step, primer elongation is performed with only one primer (forward primer) in the presence of labeled nucleotides. After PCR2, a partially double stranded probe is generated with the detection probe still free for target recognition and binding. Elongation of forward primer is blocked at the A-repeat at the 3′ end of the target-specific probe part because probe sealing is performed with a dNTP-mix consisting of A, G, C and lacking U/T.

Synthesis of sealed UPSAS may also be performed by hybridization of unsealed UPSAS with a (semi-)complementary sequence (seal) of the signal tail.

Hence, the probe (UPSAS) of this disclosure has a double function:
a. Specific detection of small target sequences with the target-specific probe part: longer sequences may be detected using a set of probes binding next to each other, and
b. Direct signal amplification of the part containing the target-specific probe: visualization of the target-specific probe(s).

This disclosure also relates to a method of detecting methylation changes and/or distinguishing methylation heterogeneity from hemi-methylation and mono-allelic methylation in situ in a sample taken from a patient comprising:
obtaining a sample from the patient,
treating the sample with adequately dosed pepsin and/or protease K and/or HCL and/or detergent and/or ethanol for permeabilization of cells and/or to remove proteins from the sample,
incubating the sample with adequately dosed bisulfite reagents in the presence of a RNase inhibitor to create non-complementary single-stranded DNA strands,
incubating the samples with specifically designed “blocking probes” and/or “RNA- or DNA-protecting probes” for at least one hour, and
incubating the sample with specifically designed “target-specific probes,” wherein a target-specific probe part is linked to a labeled compound.

With the term “methylation changes” is meant the conversion of unmethylated cytosines into methylated cytosines and vice versa.

With the term “heterogeneity” is meant a heterogenic pattern of unmethylated and methylated target genes in the same cell/sample.

With the term “hemi-methylation” is meant methylation changes that occur at one of the two DNA strands of one allele.

With the term “mono-allelic methylation” is meant methylation changes that occur at one of the two alleles per gene.

With the term “a sample of a patient” is meant a section cut from a FFPE tissue block, a fresh frozen tissue, a cell monolayer, or a smear acquired from a patient.

With the terms “adequately dosed pepsin and/or protease K and/or HCL and/or detergent” is meant the optimal amounts of pepsin or proteinase K provided, ranging from 0.001% to 10% pepsin/proteinase K, the optimal amount of HCL provided ranging from 0.005 M HCL to 4 M HCL, an optimal amount of detergent (TRITON® X-100, TWEEN®-20) provided ranging between 0.01% and 4%.

With the terms “adequately dosed bisulfite reagents in the presence of a RNase inhibitor” is meant the optimal provided concentrations of bisulfite, NaOH and RNase inhibitor ranging from 1 to 8 M bisulfite, 0.1 M to 1 M NaOH and 1:3 to 1:10000, preferentially between 1:100 to 1:1000 RNase inhibitor, respectively.

The present disclosure relates to the “target-specific probes” to detect methylation changes characterized by:

their nature as a nucleic acid or nucleic acid analog, consisting of a small target-specific part, which is able to distinguish nucleotide polymorphisms and methylation changes, and is bound to one or more labeled compound(s), which is a nucleic acid or nucleic acid analog of which the sequence(s) shows no sequence complementarity to the human genome or to the bisulfite converted sequence of the human genome and contains 10% to 100% fluorescent-labeled nucleotides.

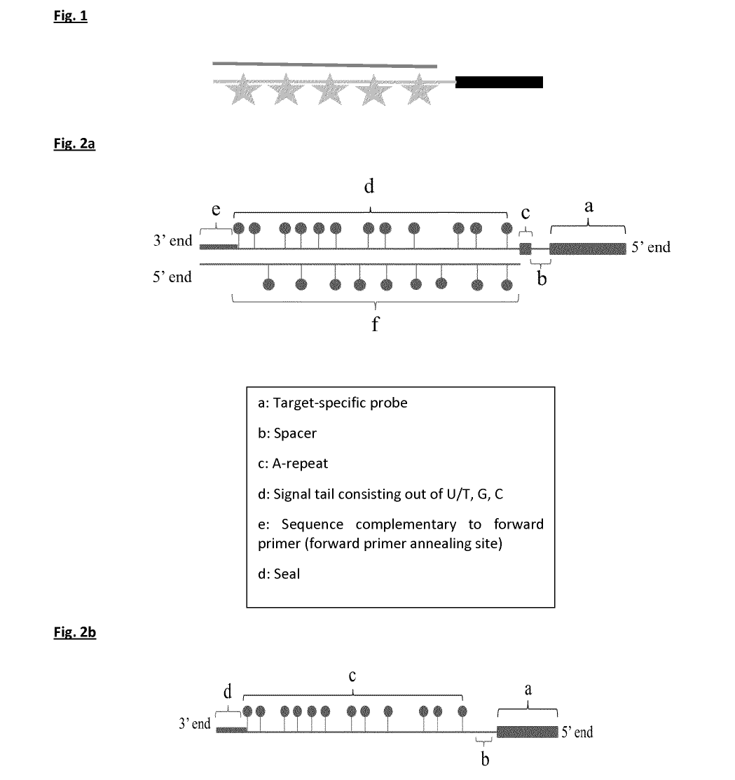

More specifically, “target-specific probes” are characterized by:
a. Their nature as a nucleic acid or nucleic acid analog, consisting of a target-specific part and a labeled compound.
b. Their number: a minimum of two target-specific probes targeting the sense and/or anti-sense strand are included, in order to increase test sensitivity.
c. The target-specific part is specifically characterized by:
   1. Their code: it should be (semi-)complementary to the bisulfite-converted target region in the DNA and/or RNA, containing a high frequency of CG sequences, through Watson-Crick base pairing. These short stretches of DNA in which the frequency of the CG sequence is higher than other regions are called “CpG islands.” CpG islands are often located around the promoters of genes. The promotor region of a gene is localized −2000 to +500, or more strict, this can be limited to −500 to +200, of the transcription start site (TSS) of the gene. A specific methylation pattern of these CpG islands in the promoter region that is characteristic for a disease state is called a “methylation marker.” A methylation pattern is defined as the order in which the methylated or unmethylated C in the CpG islands is present or absent in a target sequence. Bisulfite treatment induces unmethylated cytosine to be converted into uracil but leaves 5′ methylcytosine residues unaffected. For example, after bisulfite treatment, the methylated sequence 5′-ACmGTCCATCmGCT3′- (SEQ ID NO:3) will be converted into 5′-ACmGTUUATCmGUT-3′ (SEQ ID NO:4). The unmethylated counterpart sequence 5′-ACGTCCATCGCT-3′ (SEQ ID NO:5) will be converted in 5′-AUGTUUATUGUT-3′ (SEQ ID NO:6). The target-specific probe will specifically bind methylation markers in genes that should be detected after bisulfite treatment. For example, if the sequence of the methylation marker for a specific disease in the anti-sense strand is 5′-GAGGCmGCCmGCCmGCCmGCCmGCTGCCmGCCmGCACACTGGG ATCCmGCTCmGGCAGCA-3′ (SEQ I D NO:7), ideally, the target-specific probes for the sense and anti-sense strand should respectively be 5′-C GAA CGA ATC CCA ATA TAC GAC GAC AAC GAC GAC GAC GAC GC-3′ (SEQ ID NO:8) and 5′-CGC CGC CGC CGC CGC TAC CGC CGC ACA CTA AAA TCC GCT CGA-3′ (SEQ ID NO:9).
   2. Their size: the target-specific part of the probe is designed in such a way that it can extend between one and 10 base pairs beyond the last CpG of the intended targeted region, in order to maximize temperature difference between target-specific sequences and the mismatched sequence in a particular hybridization buffer, leading to specific hybridization to the template DNA. Preferably, the probe extends one base pair beyond the last CpG of the target region.
   3. Their linkage to “a labeled compound”: the target-specific part is covalently attached to (a) labeled compound(s) (FIG. 1). The linkage between the target-specific part and (the) labeled compound(s) is established by a linker. The linker is a 1 to 20 bp long nucleotide sequence that shows no complementarity to the bisulfite-converted sequences of the methylation marker 5′ or 3′ flanking regions, depending at which end the linker is placed, since this may interfere with mismatch detection. The linker may also be a spacer (for instance, a glycol spacer).
   d. “labeled compound” is characterized by:
   1. Its nature as a nucleic acid or nucleic acid analog.
   2. Its direct attachment to the target-specific compound (FIG. 1): the labeled complex is covalently linked to the target-specific compound by means of a linker. The linker is a nucleotide sequence of 1 to 20 bp or is a spacer (for instance, glycol spacers).
   3. Its structure:
      a. The labeled compound is large: between 200 bp and 100 kbp.
      b. The labeled compound consists of bases modified with chromogens or fluorescent dyes (for instance, biotin, FITC, Alexa Red and others).
      c. In the case where blocking of the labeled compound is pursued, a primer annealing sequence for PCR amplification is included at the 3′ end in the labeled sequence. At the 5′ end of the labeled compound, a single nucleotide may be incorporated that shows complementarity to a particular dideoxy nucleoside triphosphate (ddNTP), which is included in PCR step 2. Adding a specific ddNTP that is complementary to a nucleotide, specifically present at the 3′ end of the linker, ensures that the labeled compound and not the target-specific part is sealed during PCR step 2.
      d. The labeled compound can be sealed by a complementary sequence to prevent aspecific binding (FIG. 1).
   4. Its sequence: the labeled compound has no or a low complementarity to the human genome and the bisulfite-converted human genome.

The disclosure further relates to blocking probes characterized by:

their nature as an unlabeled nucleic acid or nucleic acid analog, having a complementary sequence to similar sequences as the target sequence, for instance, the bisulfite-converted unmethylated counterpart sequence of the target region and having a maximal bp difference of 1 kbp and preferentially of 40 bp to the target-specific part of the target-specific probes used in the same assay.

More specifically, this disclosure relates to the blocking probes characterized by:
a. Their nature as a nucleic acid or nucleic acid analog.
   b. Their number: A minimum of one and, preferentially, of two blocking probes blocking the sense and/or anti-sense strand are included, in order to increase test specificity.
   c. Their code: it should be complementary (by means of Watson-Crick base pairing) to generic sequences, to similar sequences as the target sequence and in the case where methylation changes are detected, to bisulfite-converted sequences of the DNA and/or RNA. More specifically, it blocks generic sequences, similar sequences to the target-sequence(s) and/or the bisulfite-converted unmethylated sequence(s) at the target region (in the case where the target-specific probe should detect the methylated sequence). Blocking probes thus block non-specific binding of target-specific probes to competing DNA/RNA sequences by binding these sequences. As a consequence, the target-specific probe will have the opportunity to specifically bind the target region in methylation markers in genes that should be detected (after bisulfite treatment). For example, if the sequence of a methylation marker for a disease in the anti-sense strand is 5′-GAGGCmGCCmGCCmGCCmGCCmGCTGCCmGCCmGCACACTGGGATC CmGCTCmGGCAGCA-3′ (SEQ ID NO:10), ideally, the blocking probes for the sense and anti-sense strand should, respectively, be 5′-CAA ACA AAT CCC AAT ATA CAA CAA CAA CAA CAA CAA CAA CAC-3′ (SEQ ID NO:11) and 5′-CACCACCACCACCACTACCACCACACACTAAAATCCACTCAA-3′ (SEQ ID NO:12).
   d. Their size: The blocking probes used in methylation assays are designed in such a way that they can extend between one and 10 bp and ideally between one and 2 bp beyond the last CpG of the intended targeted region. Blocking probes have the same length or a maximal size difference of 1 kbp and preferentially of 4 bp as the target-specific probes used in the same assay. The blocking probes are between 8 bp and 10 kbp and ideally between 14-200 bp long.
   e. Their distinctive character to discriminate the unmethylated target from the methylated target in methylation assays: its distinctive character to discriminate a methylated target from an unmethylated target is superior to this of the target-specific probe used in the same assay.
   f. Their freedom from labeling with chromogens or fluorescent dyes: the blocking probes are unlabeled probes.

The present disclosure further relates to unlabeled DNA-protecting probes characterized by:

their nature as an unlabeled nucleic acid or nucleic acid analog, showing (semi-)complementarity to the sequences flanking the target sequences at the 5′ end and/or 3′ end in the DNA and/or RNA, through Watson-Crick base pairing and are used to “relax” the target sequences and enhance binding of the blocking and/or target-specific probes.

More specifically, this disclosure relates to the latter unlabeled DNA-protecting probes characterized by:
a. Their nature as a nucleic acid or nucleic acid analog.
b. Their number: a minimum of one protecting probe is included, in order to hybridize to (a) sequence(s) flanking the target sequence(s) at the 5′ end and/or 3′ end.
c. Their code: they should be (semi-)complementary to the sequences flanking the (bisulfite-converted) target region at the 5′ end and/or 3′ end in the DNA and/or RNA, through Watson-Crick base pairing. More specifically, they should be (semi-)complementary to the sequence 1 to 50 bp adjacent to the 5′ end or 3′ end of the target region. They relax the RNA and (bisulfite-converted) DNA regions by forming a double-stranded structure, when they bind to the sequences flanking the target region(s). Consequently, the target region is put in a favorable position for binding the blocking probes or target-specific probes. The DNA-protecting probes will specifically bind the sequences adjacent to the target regions; in methylation assays, this is around the methylation markers in genes that should be detected (after bisulfite treatment).
d. Their size: The DNA protecting probes are between 10 and 100 kbp and ideally between 40 bp and 1 kbp. For methylation assays, they are designed in such a way that the 5′ end or 3′ end of the probe is between 1 and 20 base pairs beyond the last CpG of the intended targeted region. For example, if the sequence flanking the bisulfite-converted target sequence at the 5′ end is 5′-TGG TTA AGG TTA TTG GGG TGT TTT TGG AGA TTT AGG GGT TAA TTG GTT GGT GTT TAT ATT TAT TTG TGG GGA TTA GTG TTG TGG TGG AGA AGA GTA ATA GTA GAA GTT GGA GTT GGA GTT TGG GAG-3′ (SEQ ID NO:13); ideally the 5′ sense-protecting probe should be: 5′-C RAC TCC RAC TTC TAC TAT TAC TCT TCT CCR CCR CRA CAC TAA TCC CCA CAA ATA AAT ATA AAC ACC RAC CRA TTA ACC CCT AAA TCT CCR AAA ACA CCC CAA TAA CCT TAA CCA-3′ (SEQ ID NO:14), with R=Adenosine or guanine.
e. Their freedom from labeling with chromogens or fluorescent dyes.

This disclosure also relates to a kit comprising UPSAS and/or blocking probes and/or unlabeled DNA-protecting probes as defined above.

This disclosure relates to the usage of the latter kit to perform the method as described above.

The term “kit” refers to any manufacture (e.g., a package or a container) comprising at least one reagent/probe as described above for performing an assay/method as described above. Positive and/or negative controls can be included in the kits to validate the activity and correct usage of reagents employed in accordance with this disclosure. The design and use of controls is standard and well within the routine capabilities of those of ordinary skill in the art. The kit can be promoted, distributed, or sold as a unit for performing the methods or usages of this disclosure. Additionally, the kits can contain a package insert describing the kit and methods/usages for its use. The term “kit” is, for example, also described in WO 2009/141359 which is hereby included by reference.

This disclosure will now be illustrated by the following, non-limiting examples.

### EXAMPLES

### Example 1

### Synthesis of the UniProbe Signal Amplification System (UPSAS) Probe

Four different UPSAS probes corresponding to the Glutathione S-Transferase Pi 1 (GSTP1), hypermethylated regions in prostate cancer were designed and synthesized by PCR.

The four templates used for probe synthesis consist of five major parts (from 5′ end to 3′ end): 1) a sequence that is similar to the forward primer used in probe synthesis and sealing, 2) a part that consists of AGC nucleotides (template for signal tail), 3) a T-stretch of three nucleotides (this is included to stop probe sealing when probe sealing is performed with three nucleotides), 4) a spacer of nine nucleotides, and 5) a sequence that is complementary to the reverse primer and thus to the target-specific probe.

During the first PCR step, a forward primer (FP) with the same sequence as the sequence found at the 5′ end of the probe template and a reverse primer (RP) that will form the target-specific probe part of UPSAS, were used for amplification. A PCR reaction mix containing the forward primer, reverse primer, unlabeled dCTPs, dGTP and dATP, labeled dUTPs, unlabeled dUTP, Taq DNA Polymerase, PCR buffer, MgCl₂, nuclease-free H₂O and template, was made. Optionally, a second PCR step may be performed to (partially) seal the signal tail with a complementary sequence (“seal”).

Only the FP is used for the second PCR step; the primer is complementary to a sequence at the 3′ end of the probe. A PCR reaction mix was made containing the forward primer, unlabeled dCTPs, unlabeled dGTP, unlabeled dATP, labeled dATP, Taq DNA Polymerase, PCR buffer, MgCl₂ and nuclease-free H₂O. PCR2 was performed in the presence of only three nucleotides to stop elongation before the target-specific probe part, which is kept available for target binding. After the second PCR step, a target-specific probe, of which the signal tail carries a complementary sequence (“seal”) and thus prevents aspecific binding of the signal tail and carries additional signals, is generated.

Sealing of the unsealed probe may also be performed by incubating the unsealed probe with the “seal” allowing hybridization of the signal tail with the seal.

### Example 2

### Probe Labeling is Performed Efficiently

Probes were run on gel to confirm probe labeling and to estimate the amount of labels per probe. The probes contained at least 250 labels after PCR1 and 500 labels after PCR2.

Biotin-labeled GSTP1 probes were spotted on a nylon membrane prior to staining with 3,3′-Diaminobenzidine (DAB): spots stained dark brown, indicating that labels were not only incorporated in the probes as could be observed based on the molecular weight size on gel, but also gave strong signals.

### Example 3

### Tissue Morphology is Kept Intact after ISH Pretreatment Steps Combined with Bisulfite Conversion In Situ

3 μM formalin-fixed and paraffin-embedded (FFPE) cervical tissue sections were cut and stretched on the glass slide and deparaffinized in xylene. The sections were dehydrated two times for 5 minutes in 100% ethanol. Hereupon, the sections were incubated in 0.2 N HCl and washed with ultrapure water. The sections were treated for 28 minutes at 37° C. with porcine pepsin and washed two times for 5 minutes with ultrapure water. The sections were treated for 15 minutes with 0.1% TRITON® and washed for 3 minutes with molecular grade water afterwards. Subsequently, sections were incubated with 150 μl of a bisulfite mix (Zymo) for 4 hours. After a 15-minute desulfonation step, the samples were washed with molecular grade water and stained with hematoxylin and eosin (H&E) to evaluate conservation of the tissue morphology. Tissue morphology was evaluated by an experienced, university level pathologist, who confirmed that the morphology was kept intact.

This experiment shows that bisulfite treatment in situ and the pretreatment that should be performed prior to probe hybridization do not interfere with tissue morphology.

### Example 4

### Bisulfite Treatment Generates Single-Stranded DNA (ssDNA) In Situ

We compared DAPI staining between samples incubated with bisulfite for 1, 2, 3, and 4 hours. DAPI gives brighter signals when binding to double-stranded DNA than when binding to single-stranded DNA. As a control, we included a FFPE tissue slide that was not pretreated with bisulfite.

3 μM FFPE cervical tissue sections were cut and deparaffinized in xylene. The slides were dehydrated two times for 5 minutes in 100% ethanol. Hereupon, slides were incubated with RNase A (100 μg/ml RNase A in 2× Saline Sodium Citrate (SSC) for 45 minutes at room temperature (RT) and they were washed with 2×SSC (2×5 minutes). Slides were then treated with 0.2 M HCl and washed with ultrapure water. The slides were treated for 28 minutes at 37° C. with porcine pepsin and washed two times for 5 minutes with ultrapure water. The slides were treated for 15 minutes with 0.1% TRITON® and washed for 3 minutes with molecular grade water afterward. Subsequently, sections were incubated with 150 μl of a bisulfite solution (Zymo) for 1, 2, 3 and 4 hours. After a 15-minute desulfonation step, the slides were stained with DAPI.

Samples with a four-hour bisulfite incubation showed the highest reduction in DAPI brightness, indicating that the most ssDNA is generated after a four-hour bisulfite treatment.

### Example 5

### In Samples Pretreated According to the Described Protocol, In Situ Bisulfite Conversion is an Efficient Process

ISH pretreatment and bisulfite conversion was performed on nine FFPE skin sections; on one set of samples, an additional HCL step was performed.

Seven μM FFPE skin sections were cut and deparaffinized in xylene. Slides were dehydrated two times for 5 minutes in 100% ethanol. Hereupon, the slides were incubated in 0.2 M HCl and washed with ultrapure water and 2×SSC. The slides were then incubated for 37 minutes in 1 M NaSCN (VWR) at 80° C. and washed with ultrapure water and 2×SSC. The slides were treated for 28 minutes at 37° C. with porcine pepsin and washed two times for 5 minutes with 2×SSC. Slides were treated for 15 minutes with 0.1% TRITON® and washed for 3 minutes with molecular grade water afterward. Subsequently, sections were incubated with 150 μl of a bisulfite solution (Zymo) for 4 hours. After a 15-minute desulfonation step, the samples were washed with molecular grade water and they were scraped off in a reaction tube containing TRIZOL®. The samples were homogenized using a tissue mixer and bisulfite-converted DNA was purified. PICOGREEN® and RIBOGREEN® were used to respectively measure ds- and ssDNA concentrations in the sample extracts. The concentration of the single-stranded product significantly increased with an increasing incubation time. PCR amplification with primers specific for the bisulfite-converted regions of two genes (ACTB and TWIST (Renard et al., 2010)) was performed; efficient qPCR amplification occurred in all cases: a higher Ct (cycle threshold) value was seen in samples treated with HCL. To confirm efficient bisulfite conversion, a set of samples (incubated with bisulfite for 4 hours) was sequenced. All samples showed 99.93% to 99.97% bisulfite conversion in the amplified regions, indicating that in situ bisulfite conversion was optimal in these samples.

### Example 6

### UPSAS Probes Detect Methylation Changes in FFPE Cell Lines

Fluorescent-labeled UPSAS probes were synthesized and used to detect GSTP1 hypermethylation in breast and prostate cancer cell lines.

MCF7, LNCaP, SKBR3, BT474, PC3 and MDA-MB-231 cell lines were first tested for GSTP1 hypermethylation by MSP. In all cell lines except for MDA-MB-231, hypermethylated GSTP1 copies were detected. The amount of hypermethylated GSTP1 copies ranged between 9.7% for LNCa P and 47.1% for PC3. MDA-MB-231 was, therefore, used as a negative control for GSTP1 hypermethylation.

Four micron FFPE MCF7, LNCaP, SKBR3, BT474, PC3 and MDA-MB-231 slides were cut and deparaffinized in xylene. Slides were dehydrated in 100% ethanol. Hereupon, the slides were incubated with HCL. The slides were then washed with ultrapure water and 2×SSC. Slides were treated with porcine pepsin and washed with 2×SSC. Slides were incubated with a bisulfite solution for 3 hours at 54° C., followed by a washing step with 2×SSC. After a 15-minute desulfonation step, the slides were washed with 2×SSC. The PC3 cell line section was not treated with bisulfite but only incubated with molecular grade water. The slides were dehydrated using ethanol series (70%, 90% and 100%). After air-drying, slides were incubated overnight at 42° C. with GSTP1 methylation-specific probes. LNCaP slides were incubated with sealed or unsealed probe. Post-hybridization washes were performed in 2×SSC, 0.1×SSC and mounted for microscopic evaluation. In the majority of the cells, two dots were observed, indicating GSTP1 hypermethylation of both alleles (FIGS. 3a and 3b).

### Example 7

### Generation of Methylation Target-Specific Probes for CADM1

Target-specific probes consist of two compartments: 1) at one end, an unlabeled target-specific part that recognizes the methylation changes (in this case, CADM1 hypermethylation (Eij sink et al., 2012; Overmeer et al., 2008), and 2) at the other end, a labeled compartment whose function is to allow signalization of the target-specific part. Both compartments form the target-specific probe. The target-specific probes are synthesized by PCR. The template that is used for the generation of the probe consists of two core sequences: 1) One sequence that is identical to the target region, and 2) one random sequence of 200 bp to 100 kbp that shows low or no sequence similarity to the human genome or to the bisulfite-converted sequence of the human genome. One or two PCR steps are used for generation of the target-specific probes. During the first PCR step, a primer that is complementary to the target-specific region is used for amplification. A reaction mix containing 500 nM primer, 100 μM unlabeled dNTPs, 100 μM labeled dNTPs, 0.3 μl Taq DNA Polymerase, PCR buffer 2 μl, 1 ng template and nuclease-free H₂O was made. PCR cycling conditions are 1) Denaturation 98° C.—1 minute, 2) Cycling 1: 98° C.—20 seconds, 64° C. (sense) or 72° C. (anti-sense)—45 seconds, 72° C., 5 minutes, 20 repeats, 3) Final extension: 72° C.—1 minute, 4) Hold: 4° C., ∞ After the first PCR step, target-specific probes are generated. Optionally, a second PCR step may be performed to seal the labeled sequence with a complementary sequence. First, the probe concentration generated probe during “PCR step 1” is measured with RIBOGREEN® and 1 ng of the generated probe is used as an input for the second PCR step. One primer is used for the second PCR step, the primer is complementary to a 18 to 100 bp region at the 3′ end of the target-specific probe. A 20 μl reaction mix was made containing 500 nM primer, 200 μM unlabeled dNTPs, 0.5 [M of a ddNTP, 0.3 μl Taq DNA Polymerase, 2 μl PCR buffer, 1 ng template and nuclease-free H₂O. The PCR conditions used were: 1) Denaturation 98° C.—1 minute, 2) Cycling 1: 98° C.—20 seconds, 60° C.—45 seconds, 72° C. minutes, 20 repeats, 3) Final extension: 72° C.—1 minute, 4) Hold: 4° C., co. ddNTPs are used to stop elongation before the target-specific part, which is kept available for target binding. After the second PCR step, a target-specific probe, of which the labeled component carries a complementary sequence that seals the labeled compound and thus prevents aspecific binding of the labeled compound to a patient's DNA, is recovered.

### Example 8

### Methylation In Situ Hybridization Assay (MISH) for CADM1 Hypermethylation

MISH was demonstrated in FFPE SiHa cell lines (cervical cancer cell line). SiHa cell lines are characterized by CADM1 gene hypermethylation, a potential biomarker for squamous cervical cancer (SCC).

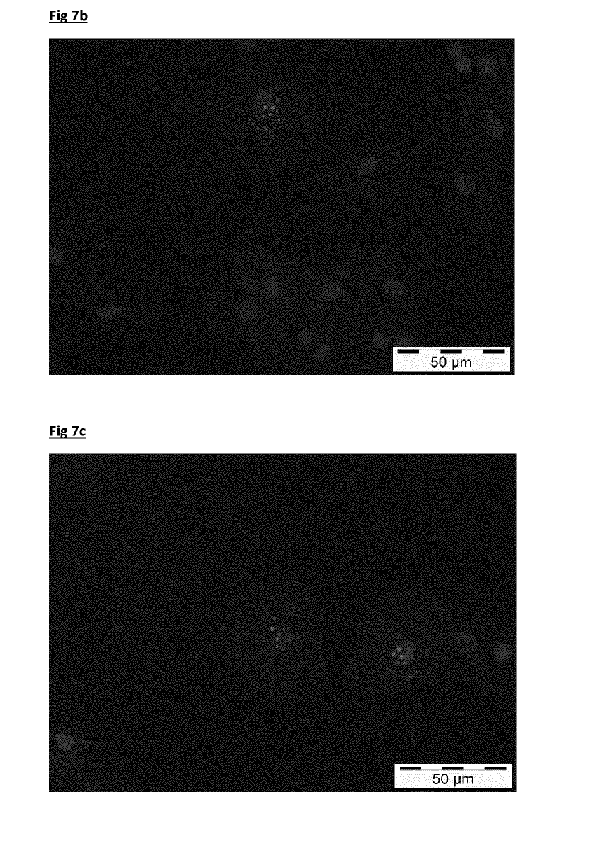

Blocking probes, specific for unmethylated sequences, target-specific probes, including a labeled compound and DNA protecting probes, are designed for the CADM1 gene. These probes can be designed for any gene. Three μM FFPE SiHa cell line sections and skin sections were cut and deparaffinized in xylene. Slides were dehydrated two times for 5 minutes in 100% ethanol. Hereupon, the slides were incubated with RNase A (100 μg/ml RNase A in 2×SSC) for 45 minutes at room temperature (RT) and washed with 2×SSC (2×5 minutes). The slides were then incubated in HCl and washed with ultrapure water and 2×SSC. Slides were treated for 37 minutes with 1 M NaSCN (VWR) at 80° C. and washed for 1 minute with ultrapure water and two times for 5 minutes with 2×SSC. Slides were treated for 28 minutes at 37° C. with porcine pepsin and washed two times for 5 minutes with 2×SSC. Slides were incubated for 5 minutes with 50% formamide at 95° C. Excessive formamide was removed and the sections were incubated with a 150 μl bisulfite solution for 4 hours at 50° C. After a 15-minute desulfonation step, the slides were washed two times for 5 minutes with 2×SSC and post-fixed with 1% formaldehyde. The slides were dehydrated using an ethanol series (70%, 90% and 100%). After air-drying, slides were incubated with blocking-probes and DNA-protecting probes for 3 hours at 45° C. Post-hybridization washes were performed in 2×SSC/0.3% NP-40 at room temperature (RT), at 42° C. to 72° C. and at RT. A second incubation with CADM1 methylation-specific probes (6 nM) was performed overnight at 45° C. Post-hybridization washes were performed in 2×SSC at RT, at 42° C. to 72° C. and at RT. The slides were then incubated with Streptavidin-FITC (1:500). Unbound streptavidin was washed away with 2×SSC. Slides were counterstained with DAPI. Consequently, hypermethylated CADM1 was evaluated in situ with a fluorescence microscope. In defined conditions, SiHa cells showed predominantly two noticeable signals per cell and skin tissue cells did not show any signal, implying specific binding of CADM1 target probes (FIGS. 6a and 6b).

### Example 9

### UPSAS Probes are Able to Specifically Detect and Visualize Single HPV73 mRNA Transcripts in Cervical Monolayers

A set of three HPV73 UPSAS probes recognizing HPV73 LI and El mRNA were designed and synthesized.

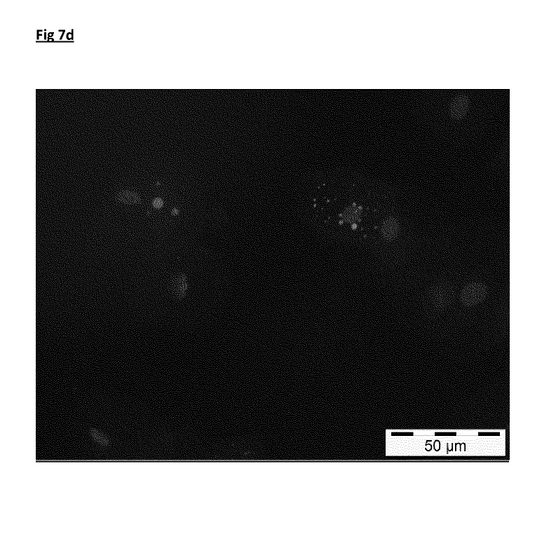

HPV73 cervical smears confirmed by qPCR using HPV73-specific primers were analyzed with the synthesized HPV73 UPSAS probes. Monolayers were fixated with paraformaldehyde (PFA), permeabilized with proteinase K and incubated with a hybridization mixture containing three HPV73 mRNA-detecting probes. Monolayers were washed and visualized with the fluorescence microscope. Individual HPV73 LI and El mRNA were adequately detected with the 40× objective (FIGS. 7a-7d).

### REFERENCES

Collins M. L., B. Irvine, D. Tyner, E. Fine, C. Zayati, C. Chang, T. Horn, D. Ahle, J. Detmer, L. P. Shen, J. Kolberg, S. Bushnell, M. S. Urdea, and D. D. Ho. A branched DNA signal amplification assay for quantification of nucleic acid targets below 100 molecules/ml. Nucleic Acids Res. 1997; 25(15):2979-84.

Eij sink, J. J. H., A. Lendvai, V. Deregowski, H. G. Klip, G. Verpooten, L. Dehaspe, G. H. de Bock, H. Hollema, W. Van Criekinge, E. Schuuring, et al. (2012). A four-gene methylation marker panel as triage test in high-risk human papillomavirus-positive patients. Int. J. Cancer 130, 1861-1869.

Larsson, C., J. Koch, A. Nygren, G. Janssen, A. K. Raap, U. Landegren, and M. Nilsson (2004). In situ genotyping individual DNA molecules by target-primed rolling-circle amplification of padlock probes. Nat. Meth. 1, 227-232.

Li, Y., Y. Miyanari, K. Shirane, H. Nitta, T. Kubota, H. Ohashi, A. Okamoto, and H. Sasaki (2013). Sequence-specific microscopic visualization of DNA methylation status at satellite repeats in individual cell nuclei and chromosomes. Nucleic Acids Research 41, e186-e186.

Nuovo, G. J., T. W. Plaia, S. A. Belinsky, S. B. Baylin, and J. G. Herman (1999). In situ detection of the hypermethylation-induced inactivation of the p16 gene as an early event in oncogenesis. Proc. Natl. Acad. Sci. U.S.A. 96, 12754-12759.

Overmeer, R. M., F. E. Henken, P. J. F. Snijders, D. Kramer Claassen, J. Berkhof, T. J. M. Helmerhorst, D. A. M. Heideman, S. M. Wilting, Y. Murakami, A. Ito, et al. (2008). Association between dense CADM1 promoter methylation and reduced protein expression in high-grade CIN and cervical SCC. J. Pathol. 215, 388-397.

Renard, I., S. Joniau, B. van Cleynenbreugel, C. Collette, C. Naome, I. Vlassenbroeck, H. Nicolas, J. de Leval, J. Straub, W. Van Criekinge, et al. (2010). Identification and Validation of the Methylated TWIST1 and NID2 Genes through Real-Time Methylation-Specific Polymerase Chain Reaction Assays for the Noninvasive Detection of Primary Bladder Cancer in Urine Samples. European Urology 58, 96-104.

Schriml L. M., H. M. Padilla-Nash, A. Coleman, P. Moen, W. G. Nash, J. Menninger, G. Jones, T. Ried, and M. Dean. Tyramide signal amplification (TSA)-FISH applied to mapping PCR-labeled probes less than 1 kb in size. Biotechniques 1999; 27(3):608-13.

1.-14. (canceled)

15. A probe comprising at least the following parts:
a first part comprising a polynucleotide that is complementary or semi-complementary to a target sequence and is similar to a reverse primer,
a second part functioning as a spacer and comprising at least one nucleotide that is not complementary to the target sequence, and
a third part comprising a polynucleotide that is not complementary to the target sequence, wherein the polynucleotides are composed of only three different types of nucleotides selected from the group consisting of A, C, G, T and U, and
wherein 10 to 100% of the nucleotides are labeled.

16. The probe of claim 15, further comprising:
a fourth part comprising a polynucleotide that is not complementary to the target sequence, but is complementary or semi-complementary to a forward primer.

17. The probe of claim 16, further comprising:
a fifth part comprising the types of nucleotides that are not chosen in the third part.

18. The probe of claim 15, wherein the third part is made double stranded by polymerase chain reaction (“PCR”).

19. The probe of claim 15, wherein the third part is made double stranded by hybridization with a sequence complementary or semi-complementary to the signal tail.

20. A method of synthesizing the probe of claim 15, the method comprising:
a polymerase chain reaction (“PCR”)step that is performed in the presence of a reverse primer that is similar to the first part of the probe, a mix of labeled and unlabeled dNTPs in order to synthesize the third part of the probe, and a template comprising out of the following parts:
optionally, a first part comprising a polynucleotide that is similar to a forward primer,
a second part comprising a polynucleotide composed of only three different types of nucleotides selected from group consisting of A, C, G, T, and U,
optionally, a third part comprising the types of nucleotides that are not chosen in the second part,
a fourth part functioning as a spacer and comprising at least one nucleotide, and
a fifth part comprising a polynucleotide that is complementary or semi-complementary to a reverse primer.

21. The method according to claim 20, wherein the third part of the probe is made double stranded by PCR and wherein the template comprises the first part and/or third part, and wherein the method further comprises:
a second PCR step performed in the presence of a forward primer and a dNTPs mix that only contain nucleotides that are complementary or semi-complementary to the three different types of nucleotides selected in the second part of the template according to the first PCR step.

22. A method of synthesizing the probe of claim 20, the method comprising:
hybridization of the signal tail with a complementary or semi-complementary sequence to the signal tail.

23. A method of specifically detecting a small target sequence, the method comprising:
utilizing the probe of claim 15 to specifically detect the small target sequence.

24. A kit comprising the probe of claim 15.

25. A method of detecting a methylation change-induced single nucleotide polymorphism in situ and/or to distinguish methylation heterogeneity from hemi-methylation and mono-allelic methylation in a sample taken from a subject, the method comprising:
treating the sample from the subject with adequately dosed pepsin and/or protease K and/or HCL and/or detergent and/or ethanol to permeabilize samples and to remove proteins from the sample,
incubating the sample with adequately dosed bisulfite reagents in the presence of a RNase inhibitor to create non-complementary single stranded DNA strands,
incubating the sample with specifically designed blocking probes and/or DNA-protecting probes for at least one 1 hour, and
incubating the sample with a specifically designed target-specific probe.

26. The method according to claim 25, wherein the specifically designed target-specific probe comprises:
a first part comprising a polynucleotide that is complementary or semi-complementary to a target sequence and is similar to a reverse primer,
a second spacer part comprising at least one nucleotide that is not complementary to the target sequence, and
a third part comprising a polynucleotide that is not complementary to the target sequence,
wherein the polynucleotides are composed of only three different types of nucleotides selected from the group consisting of A, C, G, T, and U, and
wherein 10 to 100% of the nucleotides are labeled.

27. A kit comprising:
the probe of claim 15, together with
blocking probes, and/or
DNA protecting probes.

28. The probe of claim 16, wherein the third part is made double stranded by polymerase chain reaction (“PCR”).

29. The probe of claim 16, wherein the third part is made double stranded by hybridization with a sequence complementary or semi-complementary to the signal tail.

30. The probe of claim 17, wherein the third part is made double stranded by polymerase chain reaction (“PCR”).

31. The probe of claim 17, wherein the third part is made double stranded by hybridization with a sequence complementary or semi-complementary to the signal tail.
